(19) United States
(12) Patent Application Publication
(10) Pub. No.: US 2002/0133080 A1
(43) Pub. Date: Sep. 19, 2002

(54) Layered calibration standard for tissue sampling

(76) Inventors: William Apruzzese, Everett, MA (US); Pierre Trepagnier, Medford, MA (US); Russell Gray, Brownsburg, IN (US); James Mansfield, Boston, MA (US); Christopher Lambert, Hudson, MA (US)

Correspondence Address: HELLER EHRMAN WHITE & MCAULIFFE LLP, SUITE 300, 101 ORCHARD RIDGE DR., GAITHERSBURG, MD, 20878-1917, US

(21) Appl. No.: 10/066,778

(22) Filed: Feb. 6, 2002

Related U.S. Application Data
(60) Provisional application No. 60/266,470, filed on Feb. 6, 2001.

(51) Int. Cl. A61B 6/00

(52) U.S. Cl. 600/477

(57) ABSTRACT

The invention relates to fluorescence calibration devices and methods that can mimic skin and other tissues. A calibration device of the invention comprises at least one scattering layer, which is preferably non-fluorescent, and a second layer containing one or more fluorophore. Light passes through the scattering layer and excites the fluorophore. Light emitted from the fluorophore passes back though the scattering layer and into collecting optics, which can be measured and that measurement used to correct for instrument drift.

### REFERENCE TO RELATED APPLICATIONS

This application claims the benefit of United States Provisional Patent Application Serial No. 60/266,470 filed Feb. 6, 2001.

### BACKGROUND OF THE INVENTION

1. Field of the Invention

The invention relates to devices and methods for the measurement of fluorescence spectra from biological tissue. Particularly, the invention relates to a standard for calibrating skin and tissue fluorescence measurement systems.

2. Description of Background

The skin, also known as the integumentary system, is one of the largest organs in the body. It has a surface area of 1.8 m² and makes up approximately sixteen percent of total body weight. As such, the skin represents more than a regulatory and protective barrier, it is a virtual window into the body and can report on a plethora of superficial and/or systemic alterations in health. More recently, the optical diagnostic and interventional potential of in vivo fluorescence has also begun to attract interest. Studies involving autofluorescence now include sunscreen testing, as well as laser imaging, diagnostics, drug monitoring and photodynamic therapy. Other specific applications include diagnostic testing for skin pathogens, tumors and blood/interstitial fluid analyte analysis. As a result, understanding light propagation in the skin is now viewed as fundamental to anticipating, assessing, and treating a broad spectrum of normal and abnormal conditions that affect this organ.

Optical properties of skin reflect the structure and chemical composition of the skin. When the skin surface is irradiated, part of the energy will be specularly reflected by the surface, while the rest will be refracted and transmitted into the skin. Photons transmitted into the skin will be scattered and absorbed by the skin tissue. After multiple scattering events, some of the transmitted radiation will re-emerge through the air-stratum interface into the air. This re-emergence is called diffuse reflectance. The amount of diffuse reflectance is determined by both the scattering and absorption properties of the skin tissue. Simplistically, the stronger the absorption, the less the diffuse reflection; the stronger the scattering, the larger the diffuse reflectance. Following absorption in the skin, electronically excited molecules can return to a more stable energy state by emitting a photon, which constitutes a fluorescence emission, and the reference molecule is referred to as a fluorophore. Given that there are native fluorophores localized within skin that are responsible for this fluorescence, this process is also known as autofluorescence.

Fluorescence spectra are quite sensitive to the local molecular environment of the fluorophores. Using fluorescence measurement systems that optically illuminate skin samples over a known range of frequencies and collect and measure the scattered and emitted light, normal human skin has been relatively well characterized. Clearly a complex target, skin has multiple layers, each with distinct fluorescent properties. The epidermis, composed principally of keratinocytes, is the outer, protective, nonvascular layer of the skin. It is subdivided into five layers or strata, the stratum germinatum, the stratum spinosum, the stratum granulosum, the stratum lucidum and the stratum corneum. Histologically, the stratum corneum, the outermost, keratinized layer of dead cells in the epidermis, is the most superficial layer of the skin and provides the first barrier of protection from the invasion of foreign substances into the body. Whereas the deepest epidermal layer, the stratum germinatum, provides the germinal cells necessary for the regeneration of the layers of the epidermis. It is the constant segueing from the live and actively replicating cells of the stratum germinatum to the dead cells of the stratum corneum surface that uniquely characterizes the skin. After a mitotic division a newly formed cell will undergo a progressive maturation called keratinization during which time it migrates slowly to the surface and sloughs off in a process called desquamation. Keratinocytes constitute about ninety five percent of the epidermal cells and function as a barrier, keeping harmful substances out and preventing water and other essential substances from escaping the body. The other five percent of epidermal cells are melanocytes, which manufacture and distribute melanin. Melanin, a large insoluble polymer, is a very complex absorbing material whose complete function is not understood. Generally accepted as an important factor in skin pigmentation, melanin has also been attributed with properties associated with protection from ultraviolet rays. In addition, it is known to scavenge reactive chemical species and metal ions.

The epidermis and dermis are separated by a thin layer of basement membrane to which both layers are attached. The dermis assumes the important functions of thermoregulation and supports the vascular network to supply the avascular epidermis with nutrients. The dermis is typically subdivided into two zones, a papillary dermis and a reticular layer. The dermis contains mostly fibroblasts which are responsible for secreting collagen, elastin and ground substance that give the skin its support and elasticity. Also present are immune cells that are involved in defense against foreign invaders passing through the epidermis. Given the above constituents, the dermis is gel-like and accommodates a variety of embedded structures that are common to other organs such as lymph channels, blood vessels, nerve fibers, and muscle cells, as well as unique structures like hair follicles, sebaceous glands, and sweat glands.

The fundamental principles of the optical properties of human skin is well known in the art, for example, the article published by Hardy et al., entitled “Spectral Transmittance and Reflectance of Excised Human Skin” (Journal of Applied Physics, Vol. 9, pp 257-264, 1956), describes measurements of transmission and remission of an incident beam through skin samples of various thicknesses, including both the epidermis and various amounts of dermis. The study also describes that, as the thickness of the dermis increases, transmission decreases, and becomes more diffuse, suggesting multiple scattering (as described by R. Anderson et al., in “Optical Properties of Human Skin”, The Science of Photomedicine, Plenum Press, N.Y., pgs. 147-194, 1982).

Skin or other tissue fluorescence measurements are useful for diagnosing a variety of conditions, and are often used in the cosmetics industry. Fluorescence measurements also are useful to quantitate a concentration of numerous blood analytes. In fluorescence measurement systems, acquisition of fluorescence spectra often involves a fiber optic bundle or other light illumination and collection means, which is pressed against the skin. Generally, an optics illumination/collection device or part provides excitation light to a sample and another part or device collects emission light from the sample. The position and orientation of these excitation and emission parts or devices can be adjusted to optimize detection of a desired fluorescence signal.

The ability of any signal processing technique to extract information from spectroscopic data for determination of an analyte, for example, glucose concentrations, relies heavily on the processes capability to account for nonlinearities, such a nonlinearities which can result from light penetrating skin at depths greater than 0.5 mm. The studies of Hardy et al. and Anderson et al. suggest when skin thickness exceeds 0.5 mm, the nonlinear results that must be accounted for and corrected.

Fluorescence measurement systems, therefore, should be well calibrated for the most accurate quantitation. For example, system elements such as excitation source intensity, detector efficiency, and efficiency of the optical train may change over time. Skin light scattering properties also can vary among instruments. These sources of error complicate comparisons of spectra measurements taken at different times. Stable optical calibrators that cover spectral ranges similar to those obtained from test substances are well known. Further, such calibrators exhibit standard fluorescence spectra, allowing the estimation and correction of fluorescence measurement systems. For example, Labsphere (North Sutton, N.H.) provides a line of calibration standards for the Spectralon (TM) system.

The layered geometry of the skin is vital to its function and contributes to its characteristic fluorescent spectra. The keratinized stratum corneum is highly scattering to incident irradiation. The deeper epidermal and dermal layers, based on structural elements, the presence of fluorophores and depth, also contribute to the specific spectral profiles identified with human skin. Not unexpectedly, disease processes can contribute to skin changes that are associated with corresponding shifts in structural, chemical, or histological composition. These changes are manifest as well through altered skin autofluorescence and diffuse reflectance patterns. The resulting excitation-emission profiles can also be utilized to analyze and quantify specific blood or interstitial fluid analytes (see PCT/US99/07565, PCT/US01/05323 and any U.S. counterparts). Yet the ultimate accuracy and reliability of such non-invasive optical measurement systems depends on appropriate calibration schemes that accommodate for source, sample, environmental, and temporal variations. The successful implementation of calibration methodologies is a sophisticated undertaking and requires a full understanding of measurement uncertainty, error, accuracy, precision quality, and reliability. As a rule, the accuracy of a calibration device is directly proportional to the precision of calibration with respect to the intended samples. Given that structural and biochemical factors must be taken into consideration, the design of a calibration system must anticipate skin characteristics in terms of multispectral radiation attenuation, physical morphology, and geometry. For in vivo measurement, phantoms have been used to calibrate detection systems. To date, however, available calibration devices have been unable to precisely model the spectral or fluorescence properties attributable to the complexly layered, structurally rich aspects of skin. Conventional calibration devices lack precision and accuracy because they insufficiently imitate the layered, turbid skin medium and the fluorescence properties of that medium. Thus, conventional calibration devices have not been able to characterize instruments intended for scattering, fluorescent, structured targets.

### SUMMARY OF THE INVENTION

The invention represents a significant improvement over existing devices and methods and provides accurate calibration devices that simulate any desired material or surface to produce accurate and reliable measurements.

One embodiment of the invention is directed to calibration devices that provide optical and fluorescence properties that simulate those of another material such as biological tissues and fluids. Such devices comprise at least one layer that is composed of or contains a substance of a scattering nature and another layer that contains a fluorophore. Preferably, the device is a fluorescence calibration devices comprising: at least two layers wherein a first layer comprises a scattering material, which is preferably only non-fluorescent or only slightly fluorescent, and a second layer which comprises a material having embedded fluorophores. Preferably, the fluorescence calibration device has fluorescence properties that mimic human skin.

Another embodiment of the invention is directed to methods of correcting for instrumental drift when gathering tissue fluorescence spectra, comprising the steps of: directing excitation light into a calibration artifact containing at least one scattering layer, through the scattering layer and into a fluorophore contained in another layer; exciting the fluorophore; collecting light emitted from the fluorophore; and correcting the instrumental response based on the collected light.

Another embodiment of the invention is directed to methods of calibrating a fluorescence measurement system, comprising the steps of directing excitation light into a calibration device of the invention, which excites the fluorophore and thereby collecting light emitted from the fluorophore; and calibrating the device from a measurement of the collected light.

Another embodiment of the invention is directed to methods of calibrating a fluorescence measurement system, comprising the steps of: determining a calibration target on a fluorescence measuring instrument; exciting the calibration target with amplitude modulated electromagnetic radiation; measuring electromagnetic radiation passing from the calibration target to the fluorescence measuring system; and determining a phase shift between the fluorescing amplitude modulated electromagnetic radiation, wherein the electromagnetic radiation passing from the calibration target to the fluorescence measurement system.

Another embodiment of the invention is directed to calibration devices with spectral characteristics that mimic the fluorescence properties of skin or other tissues.

The foregoing, and other features and advantages of the invention, will be apparent from the following, more particular description of the preferred embodiments of the invention, the accompanying drawings, and the claims.

### DESCRIPTION OF THE FIGURE

FIG. 1 depicts a calibration device according to one embodiment of the invention.

### DESCRIPTION OF THE INVENTION

Skin autofluorescence spectra are quite complex insofar as they are attributable to diverse fluorophores with different lifetimes, spectral properties and spatial localization. Further, spectrally active components are sensitive to dynamic fluctuations in the concentration of biological analytes. Further still, measurement changes may arise from damage or alterations to the skin, or simple heterogeneity from skin types.

It has been surprisingly discovered that an accurate emission spectrum can be created with a calibration device that calibrates multispectral optical sampling of the target being measured to resolve and correct spectral measurements for source, sample, background, environmental, geometric, and temporal variations across a plurality of samples. With devices of the present invention, the accuracy and efficiency of the measurement of optical properties from fluorescence detection systems is significantly increased.

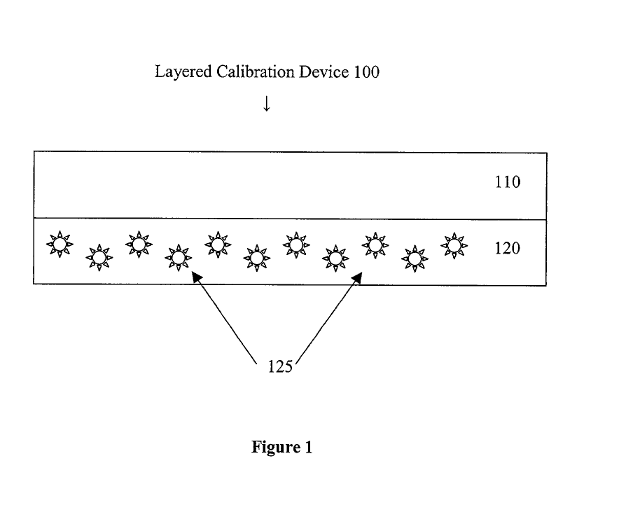

Preferred embodiments of the invention are described with reference to the FIG. 1. These preferred embodiments are discussed in the context of calibration devices that mimic human skin and tissue. Nevertheless, the invention can be practiced in the context of layered calibration devices for calibrating a wide variety of fluorescence measurement systems for a variety of purposes, not limited to glucose, and on a variety of tissues.

A layered calibration device according to an embodiment of the invention is illustrated in FIG. 1. Layered calibration device 100 comprises top layer 110 and bottom layer 120. Top layer 110 comprises a highly scattering material. Bottom layer 120 comprises a material having embedded fluorophores 125. Top layer 110 and bottom layer 120 have a desired thickness to achieve fluorescence properties that mimic a tissue such as, preferably, human skin, which is well know to those of ordinary skill in the art and can be empirically determined.

Embodiments of the invention rely on particles in the top layer to scatter light. These particles may be of a wide range of compositions and sizes. Many polymeric materials form particles of suitable size from 0.1 to 20 microns on average (i.e. at least 95% of particles falling within this range). In embodiments, narrower ranges are acceptable wherein 90% of particles have mean diameters within the range 0.2 to 1 micron, 0.3 to 1.2 microns, 1 to 20 microns 1 to 5 microns, 0.5 to 5 microns, less than 0.8 microns, or less than 1 microns. Other ranges may be determined based on the particular application by a skilled artisan. In an embodiment the particles have mean diameters that are at least 0.1 times the wavelength of light used. In another embodiment the particles have mean diameters that are at least 0.5 times, 1 time and even 2 times the wavelength of light used. In an embodiment that simulates human skin with cellular material, particles are translucent to visible light and have a mean diameter that is within 0.2 and 2 times the mean wavelength of the light used to determine light scattering.

A wide range of materials may be used for the particles. Barium sulfate is desirable for some embodiments due to its fairly even responsiveness to different wavelengths. Preferably the particles are held in place by a polymeric material. Such particles may be translucent, in which case the polymeric material may have a refractive index that differs and a particle type should be chosen having refractive index values that are at least 0.05, 0.1 or even 0.2 times different than the refractive index of the binder material. Light scattering particles having a refractive index closer to that of the binder refractive index may produce light scattering insufficient to properly simulate the human skin condition. Inorganic particles such as metal oxides typically have a higher refractive index than polymeric materials and are suitable. Other particles described in U.S. Pat. Nos. 6,255,027; 5,877,504; 4,981,882; 6,156,468 and 4,166,882 represent art known to skilled artisans and are useful.

One embodiment of the invention is directed to a method to calibrate a fluorescence measurement system. Light passes through scattering layer 110 and excites fluorophores 125 buried in a second layer 120. The emitted light then passes back though scattering layer and into the collecting optics of the system. The measured fluorescence calibration spectrum can be used to correct fluorescence instrument measurements based on the amount of light collected. The device can also be used as a standard to compare fluorescence spectra taken at different times and/or on different systems. The preferred device is lightweight, contains no environmentally harmful components, and disposable after a minimum number of uses.

A wide range of fluorophores may be used for embodiments of the invention. Representative fluorescent molecules are available from Molecular Probes (Portland, Oreg.), Eastman Kodak (Huntington, Tenn.), Pierce Chemical Co. (Rockville, Md.) and other commercial suppliers known to those of skill in the art. The fluorophore(s) may be conjugated, or may be unconjugated but immobilized within a solid layer such as a polymer. Bimanes, bodipys, and coumarins often are conjugated and are well known, as are fluorescein derivatives. Green-fluorescent Alexa Fluor 488, BODIPY FL and Oregon Green 514 dyes and the red-fluorescent Alexa Fluor 594 and Texas Red dyes, provide extremely bright signals and superior photostability and are advantageous for these reasons. In an embodiment heat stable fluors are preferred such as those described in U.S. Pat. No. 5,990,197 issued to Escano et al. In particular, monomeric infrared fluorophores such as described in U.S. Pat. Nos. 5,336,714 and 5,461,136 may be polymerized into, for example, a polyester to shift their spectral responsivity into the near infrared region. In an embodiment near infrared light of greater than 750 nm and especially greater than 800 nm light is used with a near infrared light absorbing fluorescent molecule for calibration, as near infrared has the ability to penetrate human tissue more easily and is sometimes used.

In addition to or instead of regular fluorescent reporter molecules in the calibration device, inorganic phosphors may be used. A skilled artisan is familiar with a variety of phosphors, that generally are maintained in a dry environment and which provide long decay times. This class of light emitters includes lanthanides as well, such as erbium chelates and the like. The field of semiconductor physics has developed a large number of such substances that generate emission light from excitation radiation. Some of these even act in an anti-stokes fashion, which allows a long wavelength light such as 660 nm or 880 nm light to excite a complex of lanthanide atoms and a shorter wavelength light such as 550 nm is emitted. A skilled artisan in the infrared laser arts is familiar with how to make and use these complexes, as some materials are used in this fashion to visualize infrared laser beams. This kind of emission provides an advantageous embodiment wherein the particles in layer above the light sensitive material affect excitation light differently than the emission light.

Photochemically stable fluorescent molecules are particularly desirable because of the need for reproducibility between measurements. The term “photochemically stable” in this context means that after repeated exposure a similar response can be obtained. In embodiments, the amount of radiation that is re-emitted when exposed to a constant energy source having an intensity of normal room lighting does not vary by more than five percent after at least 100, 250, 500, 1000, and even 5000 exposures. In embodiments a single exposure has a duration of 0.2 seconds, 1 second, 10 seconds and one minute.

Another embodiment of the invention is directed to a method comprising the steps: (i) directing excitation light into a calibration target containing at least one scattering layer, through the scattering layer and into a fluorophore contained in another layer; (ii) exciting the fluorophore; (iii) collecting light emitted from the fluorophore; and (iv) correcting the instrumental response based on the collected light.

Another embodiment of the invention is directed to a method of calibrating a fluorescence measurement system comprising the steps: (i) determining a calibration target on a fluorescence measuring instrument; (ii) exciting the calibration target with amplitude modulated electromagnetic radiation; (iii) measuring electromagnetic radiation passing from the calibration target to the fluorescence measuring system; and (iv) determining a phase shift between the fluorescing amplitude modulated electromagnetic radiation and the electromagnetic radiation passing from the calibration target to the fluorescence measurement system.

The term “modulated electromagnetic radiation” means that the amplitude and/or the frequency of the radiation is controlled in a reproducible way. In most embodiments the amplitude is controlled with a time varying (usually sinusoidal) signal. For example, a light emitting diode power circuit voltage may be altered to modulate the strength of the emitted light. Demodulation occurs by converting the modulation information back into a signal without the carrier light. A phase shift may be determined by sensing a time difference between the modulation frequency and the demodulated frequency. If an emission signal from a fluorophore is delayed 10 nanoseconds then the demodulated frequency, when compared to the modulating frequency will be delayed by that amount. In an embodiment an inorganic phosphor is used to generate a longer delay of at least 0.5, 1, 2 5 or even 10 milliseconds to provide larger time differences, and lower modulation frequencies. Use of lower modulation frequencies, such as less than 100 megahertz, 10 megahertz, 1 megahertz or even less than 100 kilohertz made possible by use of inorganic phosphors is desirable to keep the equipment complexity and cost down. The cost further minimized by the use of long wavelength light (greater than 600, 720, 760 or even 800 nm) generated by a photodiode or diode laser. A charge coupled device (CCD) or other two dimensional imaging device may be used as is known in the photoimaging art.

Another embodiment of the invention is directed to a method of calibrating a fluorescence measurement system, which can be applied in a technique for detecting cancer and precancerous conditions in skin, tissues and/or cells, wherein the system employs native fluorescence excitation spectroscopy.

Another embodiment of the invention is directed to a method of calibrating a fluorescence measurement system, in which the native fluorescence excitation spectra is measured at 340 nm emission with excitation over the 250 nm to 320 nm spectral region, for malignant tissues and cells are distinguishable from the corresponding excitation spectra for normal tissues and cells. Fluorescence properties are adjustable, for example, by adjusting to a desired thickness, fluorophore color (e.g. white, blue), type, concentration or distribution, fluorophore particle size, device or layer shape, or combination thereof. Alternatively or in addition, a carrier such as a clear or colored matrix or polymer can be used to adjust fluorescence properties. In a preferred embodiment, fluorescence properties are adjustable through a wavelength ranging of 200 nm to 1000 nm, and can be matched to mimic specific tissues, fluids or organs such as human skin.

Another embodiment of the invention is directed to a method of calibrating a fluorescence measurement system that provides support to a technique for detecting the presence of cancer-related, mutant proteins in samples, such as tissue samples and/or cell samples. This method can be applied to various tissues, including tissues from a part of the body, but not limited to, arteries, bladder, blood, brain, breast, capillary beds, cervix, colon, cornea, eye retina, gastrointestinal tract, gynecological tract, hair, heart, intestines, kidney, liver, lung, muscle, ovary, prostate, retinal blood vessel, skin, stomach, tumor, veins, and combinations thereof.

Another embodiment of the invention is directed to a layered calibration device that can be utilized non-invasively for calibrating sampling optics (e.g. U.S. Pat. Nos. 6,205,354, and 6,088,087), used for measuring blood volume and analyte concentration and for obtaining spectroscopic information relating to immobile tissues, such as skin. The invention provides a noninvasive calibration device for sampling optics used for determining concentration of an analyte in blood of a subject. Examples of an analyte include, but are not limited to, glucose, urea, total protein, free fatty acids, monoglycerides, diglycerides, triglycerides, creatinine, exchangeable protein associated amide protons, nucleic acids, cholesterol or combinations thereof.

Another embodiment of the invention is directed to a layered calibration device that can be used in calibrating a system for determining cell and/or organ function by measuring the blood pool clearance of a targeted agent, referred to herein as tracer (see U.S. Pat. No. 6,228,344). The cell and/or organ function can be determined by the rate these cells remove the tracer from the bloodstream. Function can also be assessed by measuring the rate the cells of interest accumulate the tracer or convert it into an active or other form. The agent, which may contain a chromophore and/or a fluorophore, may be targeted to a group of cells or organ which is a high capacity clearance system.

For agents containing chromophores and/or fluorophores, blood pool clearance is measured using a light source/photocell device that measures tissue absorbance or fluorescence in a non-target site, such as an ear lobe, finger, brain or retina. Accumulation of the tracer within the cells of interest is assessed in a similar fashion. The detection of such accumulation is facilitated by using fluorophores which emit in the near infrared wavelengths since body tissues are relatively transparent at these wavelengths.

The agent may be introduced into the patient by any suitable method, including intravenous, intraperitoneal or subcutaneous injection or infusion, oral administration, transdermal absorption through the skin, or by inhalation.

The present invention also can be used for calibrating a system used for the rapid bedside evaluation of biologic functions (see U.S. Pat. No. 6,228,344). For example, data on cardiac output, cause of hypercholesterolemia, as well as renal and hepatic function, may be obtained in less than sixty minutes at the bedside after a single intravenous injection. In accordance with one embodiment, a patient may receive a bolus injection of a plurality (e.g. 3, 4, 5, 6, etc.) of different compounds, each containing a different agent (e.g. a fluorophore).

The layered calibration device of the present invention also can be used to support system used for fluorescence detection of an agent which is cleared from the bloodstream by the kidneys or liver. Calibration of assessment of renal or hepatic function by in vivo fluorescence detection is encompassed within the invention. The invention can also be used to calibrate the monitoring of the efficiency of hemodialysis. Tumor cells or brain cells also can be targeted in accordance with the invention.

The clearance of tracers can be determined simultaneously by selecting excitation wavelengths and filters for the emitted photons. The concentration/time curves may be analyzed in real time by a microprocessor with any resulting clearance rates calculated and displayed for immediate clinical impact. In cases where unlabeled competing compounds are present (e.g. LDL, asialoglycoproteins), a single blood sample may be analyzed for the concentration of these competing compounds and the results used to calculate a flux (micromoles/minute) through the clearance pathways.

Via linear combination analysis, which is well known to those of ordinary skill in the art, calibration procedures can be implemented that interpret digitized spectra that have been subjected to mathematical algorithms and recorded as pixels. Hence, the layered calibration device of the present invention can be used to support spectral bio-imaging methods (see U.S. Pat. No. 5,784,162). The device also can be used, for example, biological research, medical diagnostics and therapeutics. The imaging methods in the are used to detect spatial organization (i.e., distribution) and to quantify cellular and tissue natural constituents, structures, organelles and administered components such as tagging probes (e.g., fluorescent probes) and drugs using light transmission, reflection, scattering and fluorescence emission strategies, with high sensitivity and high spatial and spectral resolutions.

The layered calibration device described herein can be used to calibrate a method and an apparatus for detecting the presence of a cancerous tissue, such as disclosed in U.S. Pat. No. 5,687,730. This U.S. Patent relates to a method and apparatus for detecting the presence of cancerous tissue using fluorescence. The publication relates to an apparatus for detecting the presence of abnormal tissue within a target tissue beneath the skin of a patient containing a light source producing excitation light and a calibration means.

Other embodiments and uses of the invention will be apparent to those skilled in the art from consideration of the specification and practice of the invention disclosed herein. All references cited herein for any reason, including all U.S. and foreign patents and patent applications, are specifically and entirely incorporated by reference. It is intended that the specification and examples be considered exemplary only, with the true scope and spirit of the invention indicated by the following claims.

1. A spectral calibration device comprising:
a first layer comprising an optical scattering material; and
a second layer comprising a fluorescent agent that emits radiation when subjected to an energy source,
wherein fluorescence emitted from said device provides calibration information for a spectral measurement system.

2. The device of claim 1 wherein the optical scattering material comprises particles.

3. The device of claim 2 wherein the particles comprise barium sulfate

4. The device of claim 2 wherein optical scattering of said optical scattering material can be varied by altering particle size.

5. The device of claim 1 wherein optical scattering of said optical scattering material is similar to optical scattering of a target of said spectral measurement system.

6. The device of claim 5 wherein the target is skin.

7. The device of claim 1 wherein the radiation emitted by the device is between about 200 nm and about 800 nm.

8. The device of claim 1 wherein the radiation emitted by the device is between about 300 nm and about 500 nm.

9. The device of claim 1 wherein the energy source is visible light.

10. The device of claim 1 wherein the fluorophore is photochemically-stable.

11. The device of claim 10 wherein the photochemically stable fluorophore is a fluorophore that emits an amount of radiation when exposed to a constant energy source wherein said amount does not vary by more than five percent after at least 250 exposures to said source.

12. The device of claim 10 wherein the photochemically stable fluorophore is an inorganic phosphor.

13. The device of claim 1 wherein the first layer is proximal to said energy source and the second layer is distal to said energy source, such that radiation passes through said first layer before reaching the agent.

14. The device of claim 1 wherein the spectral measurement system provides a glucose level determination for a patient.

15. A method of calibrating a spectral measurement comprising:
directing excitation radiation to a calibration device comprising at least one optical scattering layer and at least one other layer containing at least one fluorophore, wherein optical radiation emitted from said device provides calibration information;
collecting spectral radiation emitted from the device; and
calibrating the spectral measurement with the calibration information collected.

16. The method of claim 15 wherein the spectral measurement is fluorescence.

17. The method of claim 15 wherein the spectral measurement is indicative of a glucose level of a patient.

18. The method of claim 15 wherein the excitation radiation comprises visible light.

19. The method of claim 15 wherein the scattering layer comprises particles of barium sulfate.

20. The method of claim 15 wherein the fluorophore comprises a photochemically stable inorganic phosphor.

21. The method of claim 15 wherein the spectral radiation emitted is between about 200 nm and 800 nm.

22. The method of claim 15 wherein the spectral radiation emitted is between about 300 nm and 500 nm.

23. The method of claim 15 wherein the emitted radiation is collected with a CCD camera.

24. The method of claim 15 wherein the calibration information is determined from the amount of radiation detected from the fluorophore.

25. A method of calibrating a fluorescence measurement comprising:
selecting a calibration target on a fluorescence measuring instrument;
exciting the calibration target with modulated electromagnetic radiation;
detecting spectral radiation emitted from the calibration target; and
demodulating the spectral radiation detected to generate a difference signal that is indicative of a phase shift between the modulated electromagnetic radiation and the spectral radiation emitted from the calibration target.

26. The method of claim 25 wherein the calibration target is selected from the group consisting of tissues of arteries, bladder, blood, brain, breast, capillary beds, cervix, colon, cornea, eye retina, gastrointestinal tract, gynecological tract, hair, heart, intestines, kidney, liver, lung, muscle, ovary, prostate, retinal blood vessel, skin, stomach, tumor, veins, and combinations thereof

27. A method of correcting for drift of an optical instrument comprising:
directing excitation radiation to a calibration device comprising at least one scattering layer and at least one other layer that contains a fluorophore;
collecting emitted radiation from the device and determining a correction signal; and
calibrating the optical instrument from the correction signal.

28. A device for calibrating a glucose level detection instrument comprising an outer layer containing an optically scattering material and an inner layer comprising a fluorophore, wherein visible radiation emitted through said outer layer impacts said fluorophore which thereby fluoresces and provides a calibration correction measurement to the glucose level detection instrument which provides an accurate glucose level determination for a patient.
